(19) United States
(12) Patent Application Publication
(10) Pub. No.: US 2014/0326251 A1
(43) Pub. Date: Nov. 6, 2014

(54) BREAST SEPARATOR AND METHODS THEREOF

(71) Applicant: Theresa Trentacosta, Tinton Falls, NJ (US)

(72) Inventor: Theresa Trentacosta, Tinton Falls, NJ (US)

(21) Appl. No.: 13/887,214

(22) Filed: May 3, 2013

(51) Int. Cl. A61F 5/40

(52) U.S. Cl. CPC A61F 5/40
USPC 128/845

(57) ABSTRACT

A breast separator is provided that may comprise a top side; a bottom side; and a pair of lateral sides extending between the top side and the bottom side, the pair of lateral sides having concave edges partially surrounding openings, the openings adapted to receive a pair of breasts of a user; wherein the top side, the bottom side, and the pair of lateral sides are adapted to prevent the pair of breasts of the user from substantially moving outside of the openings when the user is laying on at least one of their side and their stomach.

### BACKGROUND OF THE INVENTION

1. Field of the Invention

Embodiments of the present invention generally relate to a breast separator and methods thereof. More specifically, embodiments of the present invention relate to a breast separator for placement between the breasts of a user for reducing and/or eliminating the discomfort and/or potential wrinkling of the breast skin or the chest area above, below, or adjacent the breasts, when the user sleeps and/or lays on their side or stomach.

2. Description of the Related Art

Many individuals with breasts desire to sleep on their stomachs and or sides. When an individual with breasts sleeps on their stomach or side, however, the breasts and/or the chest area around the breasts may wrinkle, fold, bunch up, and/or collapse. When the breasts and/or the chest area around the breasts wrinkle, fold, bunch up, and/or collapse, it is often uncomfortable, irritating, and/or painful to these individuals. In addition to causing discomfort to the individual, wrinkling of the breasts and/or the chest area around the breasts may often lead to permanent wrinkling of the skin of the breasts and/or the chest area, or deforming of the breasts and/or chest area, which may detract from the general aesthetic appearance of the breasts and/or chest area. Many individuals desire aesthetically pleasing breasts. Some of these individuals have a natural tendency or desire to sleep on their sides or stomachs. To avoid affecting the aesthetic appearance of their breasts and/or avoid discomfort, many of these users force themselves to sleep in different positions than their natural inclination or desires. These users often attempt to avoid side and/or stomach sleeping altogether.

Attempts to make stomach sleeping more comfortable for those with breasts have included using wedges for placement on a user's stomach. These wedges fail to solve the problem, however, because they do not effectively restrict lateral movement of the breasts, do not fully support the breasts, and do not allow the user to sleep on their side.

Thus, there is a need for an improved breast separator for reducing and/or eliminating the discomfort and/or potential wrinkling of the breast and/or chest skin that may occur when a user sleeps on their side or stomach.

### SUMMARY

Embodiments of the present invention generally relate to a breast separator, that may comprise a top side; a bottom side; and a pair of lateral sides extending between the top side and the bottom side, the pair of lateral sides having concave edges partially surrounding openings, the openings adapted to receive a pair of breasts of a user; wherein the top side, the bottom side, and the pair of lateral sides are adapted to prevent the pair of breasts of the user from substantially moving outside of the openings when the user is laying on at least one of their side and their stomach.

In exemplary embodiments, a breast separator, may comprise a top side; a bottom side; a pair of lateral sides extending between the top side and the bottom side, the pair of lateral sides having concave edges partially surrounding openings, the openings adapted to receive a pair of breasts of a user; a securing member adapted to secure the breast separator around the outside of at least one of the pair of breasts; and wherein the top side, the bottom side, and the pair of lateral sides are adapted to prevent the pair of breasts of the user from substantially moving outside of the openings when the user is laying on at least one of their side and their stomach.

In exemplary embodiments, a method for securing breasts may comprise providing a breast separator comprising: a top side; a bottom side; a pair of lateral sides extending between the top side and the bottom side, the pair of lateral sides having concave edges partially surrounding openings, the openings adapted to receive a pair of breasts of a user; and a securing member adapted to secure the breast separator around the outside of at least one of the pair of breasts; wherein the top side, the bottom side, and the pair of lateral sides are adapted to prevent the pair of breasts of the user from substantially moving outside of the openings when the user is laying on at least one of their side and their stomach; and placing the breast separator between a pair of breasts of a user.

### BRIEF DESCRIPTION OF THE DRAWINGS

So the manner in which the above recited features of the present invention can be understood in detail, a more particular description of embodiments of the present invention, briefly summarized above, may be had by reference to embodiments, which are illustrated in the appended drawings. It is to be noted, however, the appended drawings illustrate only typical embodiments of embodiments encompassed within the scope of the present invention, and, therefore, are not to be considered limiting, for the present invention may admit to other equally effective embodiments, wherein:

FIG. 1 depicts a perspective view of a breast separator in accordance with embodiments of the present disclosure;

FIG. 2 depicts a front view of the breast separator depicted in FIG. 1 in use in accordance with embodiments of the present disclosure;

FIG. 3 depicts front view of a breast separator with a securing member in accordance with embodiments of the present disclosure; and

FIG. 4 depicts a flowchart of a method of using a breast separator in accordance with embodiments of the present disclosure.

The headings used herein are for organizational purposes only and are not meant to be used to limit the scope of the description or the claims. As used throughout this application, the word may is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

### DETAILED DESCRIPTION

Embodiments of the present invention generally relate to a breast separator and methods thereof. More specifically, embodiments of the present invention relate to a breast separator for placement between the breasts of a user for reducing and/or eliminating the discomfort and/or potential wrinkling of the breast skin and/or the chest skin around the breasts, when the user sleeps on their side or stomach. The chest skin may comprise any skin in the chest area. The chest area may comprise the area above, below, between, to the side, or adjacent the breasts, or any area of the chest outside of the breasts.

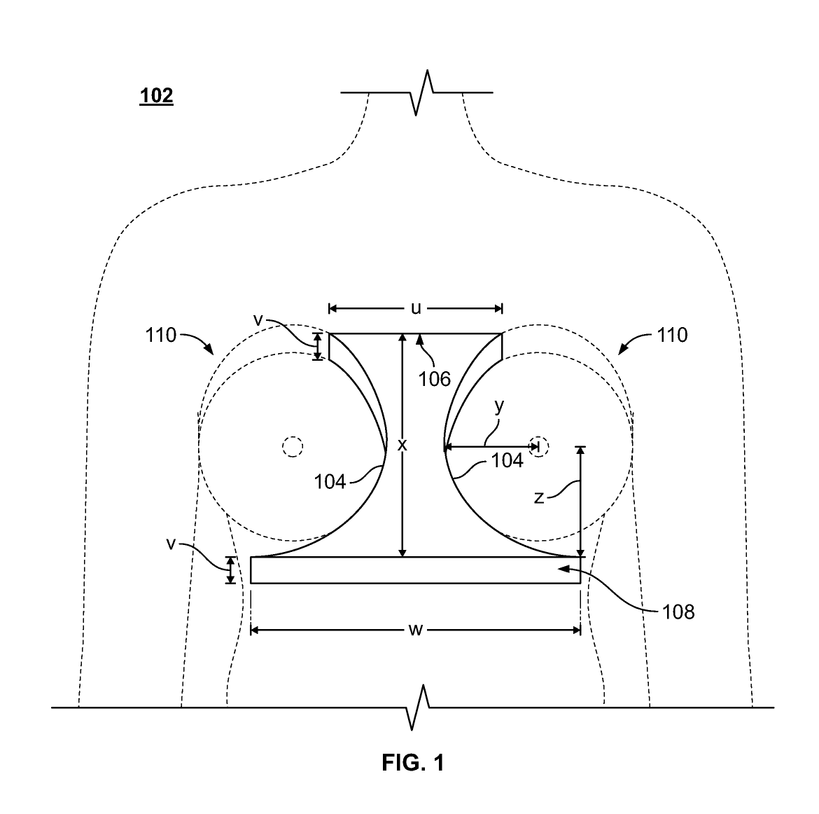

FIG. 1 depicts a perspective view of a breast separator 102 in accordance with embodiments of the present disclosure. A breast separator 102 may generally comprise a top side 106, a bottom side 108, and a pair of lateral sides 104 extending between the top side 106 and the bottom side 108. The top side 106 and bottom side 108 may generally be flat, and may be joined by lateral sides 104. The lateral sides 104 may generally be C-shaped or concave, and may partially encircle a pair of openings 110 adapted to receive a pair of breasts. While the edges in the figures are depicted as flat, it is contemplated that curved edges may be used to enable breast separator 102 fits comfortably against the skin of the user. For example, any/all of the edges may be beveled, double beveled, chamfered, demi bullnosed, full bullnosed, half bullnosed, double demi bullnosed, ogge bullnosed, and/or the like.

The breast separator 102 may comprise any materials suitable for embodiments of the present disclosure. In one embodiment, the breast separator 102 may comprise at least one of a porous foam, a non-porous foam, and a medical grade plastic gel compound. For example, the breast separator 102 may comprise a foam made from a solid, spongy cellular material produced by the reaction of polyester with a diisocyanate while carbon dioxide is liberated by the reaction of a carboxyl with the isocyanate. In this embodiment, polyester resin may react with a compound while carbon dioxide (CO₂) is simultaneously released by another reaction. This gas creates open pockets within the polyurethane that, in turn, makes the material soft and light. In another example, the breast separator 102 may comprise a medical gel, a thick, rubber-like material, and/or medical grade plastic gel compound. In some embodiments, when a gel is used, the gel may encased in a casing wherein the gel and may be heated or cooled/frozen within the casing prior to use, to provide a heating or cooling sensation to the skin of the user. In embodiments, the casing may be sealed to prevent the gel from escaping and may comprise a plastic.

A breast separator 102 may generally comprise a material that is comfortable against the skin of the user when placed between the user's breasts. The material may be comfortable to lay on and sleep with, but also may have a degree of rigidity to allow the breast separator 102 to keep the breasts of the user separated and to substantially prevent wrinkling of the breasts and/or the chest area around or adjacent to the breasts. For example, in addition to foam and medical grade plastic gel, the breast separator 102 may comprise a fabric stuffed with a stuffing, such as cotton or down. Further examples of acceptable materials may include flexible plastic, rubber, leather, cork, textiles, microfiber, sponge, elastic, and/or the like. The breast separator 102 may comprise one layer or may comprise more than one layer joined together with an adhesive or the like. For example, the breast separator 102 may comprise a soft bottom layer, a rigid center layer, and soft lateral layers.

A breast separator 102 may also be disposable and made of disposable and/or recyclable materials. For example, a breast separator 102 may comprise recyclable paper, plastic, cardboard, fiberboard, and/or the like. In some embodiments, a soft cover (not shown) may be used to cover the breast separator 102 and make it more comfortable for the user to use, especially when the materials used to make the breast separator 102 are somewhat coarse. The cover may be disposable and/or washable/reusable.

While depicted in the Figures as a single piece, the breast separator 102 may comprise more than one piece. For example, the breast separator 102 may comprise a top piece and a bottom piece (not shown) that are adapted to be detachably attached. When the breast separator 102 is separated into two pieces it may be easier to store and/or transport than the one piece embodiment. In addition, the breast separator 102 may be foldable about an axis (not shown) to allow for ease of storage and/or transportation.

In exemplary embodiments, the dimensions of the breast separator 102 may be adapted to separate breasts of different volumes, cup sizes, and shapes. For example, the breast separator 102 may comprise dimensions adapted to separate breasts of cup size AA, A, B, C, D, DD, DDD, E, F, G, H, I, J, K, L, M, N. The breast separator 102 may also comprise dimensions adapted to separate non-pendulous or pendulous breasts, artificial breasts, natural breasts, and breasts of unequal sizes, volumes, shapes and/or the like. In exemplary embodiments, a portion of the top side 106 may comprise a width U adapted to fit on a top side of the breasts of a user. The width U of the top side 106 may be less than the width W of the bottom side 108, or they may be equivalent. A portion of the bottom side 108 may be adapted to fit on a bottom side of the breasts of a user. In exemplary embodiments, the width U of the top side 106 may comprise between 1″ to 7″. For example, the width U of the top side 106 may comprise 3.5″. In exemplary embodiments, the width W of the bottom side 108 may comprise between 1″ to 12.″ For example, the width W of the bottom side 108 may comprise 6″. The thickness V of the breast separator 102 may comprise between 0.25″ and 3″. For example, the thickness V of the breast separator 102 may comprise 1″. When measured to the approximate center point of the opening 110 for the breast of the user, the lateral depth Y may comprise between 0.25″ and 3″ and the vertical depth Z may comprise between 0.25″ and 3″. For example, the lateral depth Y may comprise 1″ and the vertical depth Z may comprise 1″. The height X of the breast separator 102 may comprise between 1″ and 8″. For example, the height X may comprise 4″. Although specific dimensions and measurements are provided herein, any dimensions suitable for embodiments of the present disclosure are contemplated. For example, the dimensions of the breast separator 102 may be adapted to receive any size, shape, volume, or mass or breast.

In exemplary embodiments, the lateral sides 104 may be generally “C” and reverse “C” shaped and may form openings 110 for accepting the breasts of a user. The lateral sides 104 may comprise degrees of concavity consistent with the present disclosure. For example, the lateral sides 104 of the breast separator 102 may comprise a degree of concavity to accept breasts of the same size, shape, volume, mass, or breasts of different sizes, shapes, volumes, and masses. The breast separator 102 may comprise a uniform thickness, and both lateral sides 110 may comprise identical dimensions facing in opposite directions.

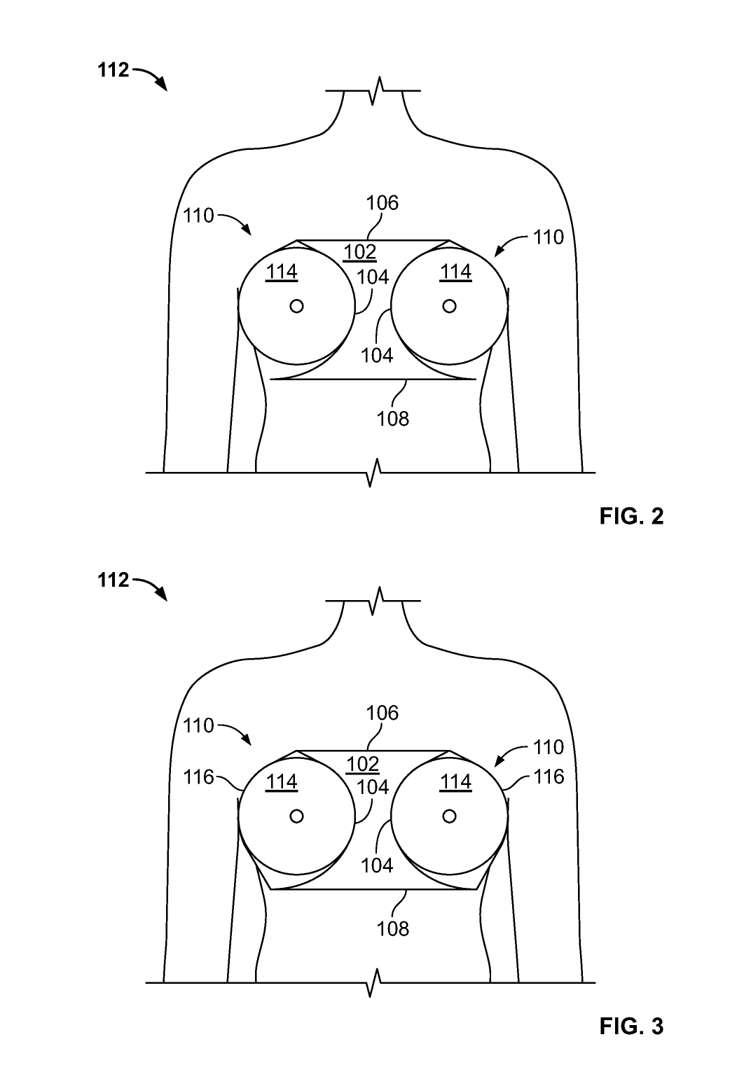

In exemplary embodiments, the breast separator 102 may be adapted to accept two breasts that are unequal in size, shape, volume, mass and/or the like. Different sets of dimensions may be used for each lateral side 110 and/or the top side 106 and bottom side 108. For example, a user may have two breasts of different sizes, such as B cup and C cup. In this example, one of the lateral openings 110 may be adapted to accept the B sized breast and one of the openings may be adapted to accept the C sized breast. As an additional example, a user may have one pendulous breast and one non-pendulous breast. In this example, the length and thickness of the bottom side of the breast separator 102 may be different for each breast. Similarly, the length and thickness of the top side 106 may be different from the bottom side, and the length and thickness of the lateral sides of the top side 106 may be different. An example of a breast separator 102 in use may be seen in FIG. 2.

Referring now to FIG. 2, a breast separator 102 is shown in use on a user 112. In exemplary embodiments, the user 112 may place the breast separator 102 in-between the breasts 114 of the user 112. As described above, the breast separator 102 may comprise lateral sides 104 of a concave shape forming openings 110 for accepting the breasts 114 of the user. When the breast separator 102 is in place between the breasts 114, the breast separator 102 may be adapted to substantially prevent the breasts and or the chest area around the breasts, from wrinkling, folding, collapsing, or the like, when the user is laying on their side or on their stomach. Although depicted as spheres, it is contemplated that many breasts 114 are not spherical, and the breast separator 102 may be adapted to accept breasts of different sizes and shapes. In some embodiments, inner edges of the lateral sides 104 may be angled to support one or more of the breasts 114, or may be flat. In some embodiments, the inner edges of the lateral sides underneath the breasts 114 may comprise a notch, a beveled edge, and a cup, or a curved portion for accepting a portion of one or more of the breasts 114. The breast separator 102 may be frictionally fit between the breasts 114. In some embodiments, as shown in FIG. 3, one or more securing members may be used to secure the breast separator 102 the breasts 114 of the user 112.

Referring now to FIG. 3, a front view of a breast separator 102 with securing members 116 in accordance with embodiments of the present disclosure is shown. In exemplary embodiments, after a breast separator 102 is placed between the breasts 114 of a user, the breast separator 102 may be further secured around the outside of the breasts 114 with one or more securing members 116. The securing members 116 may comprise a material that is comfortable for the user to wear, such as a soft fabric, elastic, strap, or the like. For example, the securing members 116 may comprise a strap encased in a soft material such as a fur. The securing members 116 may be adapted secured to the top side 106 and/or the bottom side 106 by stitching, adhesive, or the like. The securing members 116 may also be releaseably secured to the breast separator 102 with buckles, hook and loop fasteners, snaps, buttons, or the like. By way of example, the securing members 116 may comprise straps attached to the bottom side 108 of the breast separator 102 with a stitching and securable to the top side 106 of the breast separator 102 with a hook and loop fastener. Although depicted as a single piece, ach of the securing members 116 may comprise two or more pieces. For example, a securing member 116 may comprise a portion attached to the top side 104, a portion attached to the bottom side 108, and an attachment means for securing the two portions together. The attachment means (not shown) may be any attachment means suitable for embodiments of the present disclosure, for example, buckles, hook and loop fasteners, snaps, buttons, and/or the like. In some embodiments, one or more alternative securing members (not shown) may be included. The alternative securing members may include straps adapted to fit around the back of the user 112. The straps may attach two opposing sides of the top side and/or two opposing sides of the bottom side.

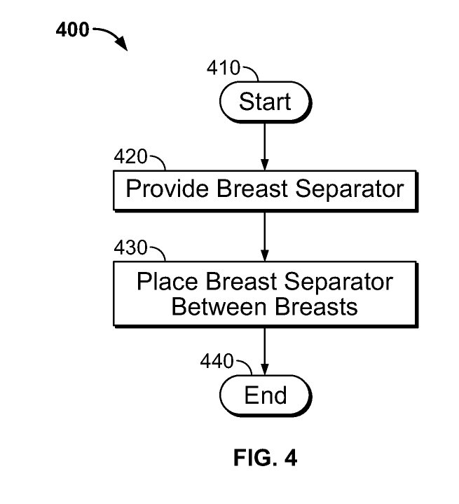

In alternative embodiments, the breast separator 102 may be adapted for use by post-surgical breast enhancement surgery patients to aid in the healing process. In these embodiments, individuals who have undergone breast enhancement surgeries, for example, breast augmentations, breast lifts, breast revisions, or any type of surgery performed on the breast or breasts, may be able to use the breast separator 102 to aid in the healing process after surgery and/or to reduce the likelihood an infection will occur. The breast separator 102 may comprise medical-grade materials to aid in the healing process. The breast separator 102 may comprise a partial disposable portion, such as a gauze, a cover, a padded section, or the like and a partial non-disposable portion adapted to provide support to the disposable portion. In some embodiments, the breast separator 102 may comprise disposable materials, and the entire breast separator 102 may be disposable after use. A partial or entirely disposable breast separator 102 may be beneficial to patients using the breast separator 102 to aid in the healing process because the breast separator may be contacted by bodily fluids, such as blood or the like, as the result of the surgery or potential post-surgery infections. The breast separator 102 may also comprise an absorbent portion or may be completely absorbent. For example, the breast separator 102 may comprise an absorbent portion disposed where the breast separator 102 will come into contact with the breasts, adjacent areas where the breast separator 102 may come into contact with the breasts, or on the entire outer surface of the breast separator 102. In some embodiments, the entire breast separator 102 may comprise an absorbent material. A method of using the breast separator 102 is described in FIG. 4.

Referring now to FIG. 4, a flowchart of a method 400 of using a breast separator in accordance with embodiments of the present disclosure is depicted. The method begins at step 410. At step 420, a breast separator 102, such as the examples disclosed herein with respect to FIGS. 1-3 is provided. For example, the breast separator may generally comprise a top side, a bottom side, lateral dies, and openings. In some embodiments, the breast separator may comprise a securing member.

In exemplary embodiments, at step 430, the breast separator may be placed between the breasts of the user such that the breasts of the user fit within the openings formed by the lateral sides of the breast separator. After the breast separator is placed, it may be secured between the breasts by way of friction fit between the breasts. In some embodiments, if securing member is provided, the securing member may be used to secure the breast separator around the outside surface of the breasts of the user and/or around the back of the user. If the securing member is present only on one side of the breast separator, the securing member may be secured only to that side. Alternatively, if the securing member is present on both sides of the breast separator, the securing member may be secured on both sides. After the breast separator is in place and optionally secured with a securing member, the user may then sleep on their stomach or side and the breast separator may be adapted to prevent or substantially reduce wrinkling, folding, collapsing, or the like of the breasts and/or the chest area around or adjacent the breasts, and/or substantially prevent the breasts from touching each other. After the user places the breast separator between their breasts and falls asleep on their back and/or stomach, the method ends at step 440.

While the foregoing is directed to embodiments of the present invention, other and further embodiments of the invention may be devised without departing from the basic scope thereof. For example, although numerous embodiments having various features have been described herein, combinations of such various features in other combinations not discussed herein are contemplated within the scope of embodiments of the present invention.

What is claimed is:

1. A breast separator, comprising:
a top side;
a bottom side; and
a pair of lateral sides extending between the top side and the bottom side, the pair of lateral sides having concave edges partially surrounding openings, the openings adapted to receive a pair of breasts of a user;
wherein the top side, the bottom side, and the pair of lateral sides are adapted to prevent the pair of breasts of the user from substantially moving outside of the openings when the user is laying on at least one of their side and their stomach.

2. The breast separator of claim 1, wherein at least one of the top side and the bottom side is flat.

3. The breast separator of claim 1, wherein the lateral sides are C-shaped.

4. The breast separator of claim 1, wherein at least one of the top side, the bottom side, and the pair of lateral sides comprises a beveled edge.

5. The breast separator of claim 1, wherein the breast separator comprises at least one of a porous foam, a non-porous foam, and a medical grade plastic gel compound.

6. The breast separator of claim 1, wherein the breast separator comprises a foam made from a solid, spongy cellular material produced by the reaction of polyester with a diisocyanate while carbon dioxide is liberated by the reaction of a carboxyl with the isocyanate.

7. The breast separator of claim 1, wherein the breast separator comprises a gel contained within a casing, the gel adapted to be frozen and contained within the casing.

8. The breast separator of claim 1, wherein a width of the top side is between 1″ to 7″;
wherein a width the bottom side is between 1″ to 12″;
wherein a thickness of the breast separator is between 0.25″ and 3″; and
wherein a height of the breast separator is between 1″ and 8″.

9. The breast separator of claim 1, wherein each of the lateral sides comprise different dimensions adapted to accept breasts of different sizes.

10. The breast separator of claim 1, further comprising:
a securing member adapted to secure the breast separator around the outside of at least one of the pair of breasts.

11. The breast separator of claim 10, wherein the securing member comprises a strap.

12. The breast separator of claim 10, wherein the securing member is attached to at least one of the top side and the bottom side.

13. The breast separator of claim 12, wherein the securing member is attached with at least one a stitching, adhesive, buckles, hook and loop fasteners, snaps, and buttons.

14. A breast separator, comprising:
a top side;
a bottom side;
a pair of lateral sides extending between the top side and the bottom side, the pair of lateral sides having concave edges partially surrounding openings, the openings adapted to receive a pair of breasts of a user;
a securing member adapted to secure the breast separator around the outside of at least one of the pair of breasts; and
wherein the top side, the bottom side, and the pair of lateral sides are adapted to prevent the pair of breasts of the user from substantially moving outside of the openings when the user is laying on at least one of their side and their stomach.

15. The breast separator of claim 14, wherein the breast separator comprises at least one of a porous foam, a non-porous foam, and a medical grade plastic gel compound.

16. The breast separator of claim 14, wherein the securing member comprises a strap.

17. The breast separator of claim 14, wherein the breast securing member is attached to at least one of the top side and the bottom side.

18. The breast separator of claim 14, wherein the securing member is attached with at least one a stitching, adhesive, buckles, hook and loop fasteners, snaps, and buttons

19. A method for securing breasts, comprising:
providing a breast separator comprising:
a top side;
a bottom side;
a pair of lateral sides extending between the top side and the bottom side, the pair of lateral sides having concave edges partially surrounding openings, the openings adapted to receive a pair of breasts of a user; and
a securing member adapted to secure the breast separator around the outside of at least one of the pair of breasts;
wherein the top side, the bottom side, and the pair of lateral sides are adapted to prevent the pair of breasts of the user from substantially moving outside of the openings when the user is laying on at least one of their side and their stomach; and
placing the breast separator between a pair of breasts of a user.

20. The method of claim 19, further comprising:
securing the securing member around the outside of at least one of the pair of breasts.
